(19) United States
(12) Plant Patent Application Publication
(10) Pub. No.: US 2013/0074237 P1
(43) Pub. Date: Mar. 21, 2013

(54) Gaillardia plant named 'DGAL906'

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 13/200,185

(22) Filed: Sep. 19, 2011

(51) Int. Cl. A01H 5/00

(52) U.S. Cl. PLT/431

(57) ABSTRACT

A new and distinct Gaillardia cultivar named ‘DGAL906’ is disclosed, characterized by a distinctive crimson red flower, somewhat compact plant habit, good branching and abundant flowering. The new variety begins blooming early in the season and flowers over a long period of time. The new variety is a Gaillardia, normally produced as an outdoor garden or container plant.

### LATIN NAME OF THE GENUS AND SPECIES

Gaillardia pulchella

### VARIETY DENOMINATION

‘DGAL906’

### BACKGROUND OF THE INVENTION

The new Gaillardia cultivar is a product of a planned breeding program conducted by the inventor, Gavriel Danziger, in Moshav Mishmar Hashiva, Israel. The objective of the breeding program was to produce new Gaillardia varieties for ornamental commercial applications. The cross resulting in this new variety was made during August 2005.

The seed parent is the unpatented, proprietary seedling variety referred to as Gaillardia pulchella ‘GAI-45’. The pollen parent is unknown, as the crossing resulting in ‘DGAL906’ was an open pollination, with unidentifiable pollen parents. The new variety was discovered in July 2006 by the inventor in a group of seedlings resulting from the 2005 crossing, in a research greenhouse in Moshav Mishmar Hashiva, Israel.

Asexual reproduction of the new cultivar ‘DGAL906’ by vegetative cuttings was first performed at a research greenhouse in Moshav Mishmar Hashiva, Israel in February 2007 and has shown that the unique features of this cultivar are stable and reproduced true to type in successive generations.

### SUMMARY OF THE INVENTION

The cultivar ‘DGAL906’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DGAL906’ These characteristics in combination distinguish ‘DGAL906’ as a new and distinct Gaillardia cultivar:

1. Somewhat compact habit

2. Highly floriferous

3. Long flowering season

4. Good branching

5. Early blooming

6. Unique crimson red flower color

Plants of the new cultivar ‘DGAL906’ are similar to plants of the seed parent, Gaillardia pulchella ‘GAI-45’ in most horticultural characteristics, however, plants of the new cultivar ‘DGAL906’ produce more flowers and branches on a more compact plant than ‘GAI-45’.

### COMMERCIAL COMPARISON

Plants of the new cultivar ‘DGAL906’ are comparable to the variety Gaillardia pulchella ‘Torch Red Embers’, patent status unknown after reasonable search by the applicant. The two Gaillardia varieties are similar in most horticultural characteristics, however, the new variety ‘DGAL906’ differs in having more flowers per plant, a slightly different color pattern to the flowers and a more compact, and spreading plant habit. Additionally, ‘DGAL906’ has semi-double flowers, compared to the double flowers of ‘Torch Red Embers’.

Plants of the new cultivar ‘DGAL906’ can also be compared to the commercial variety Gaillardia ‘BALTORYEL’ U.S. Plant Pat. No. 15,966. These varieties are similar in most horticultural characteristics, however plants of ‘DGAL906’ produce semi-double, red colored flower, whereas plants of ‘BALTORYEU’ produce double, solid yellow flowers. Additionally, ‘DGAL906’ has more flowers per plant, and a more compact, spreading plant habit.

### BRIEF DESCRIPTION OF THE PHOTOGRAPH

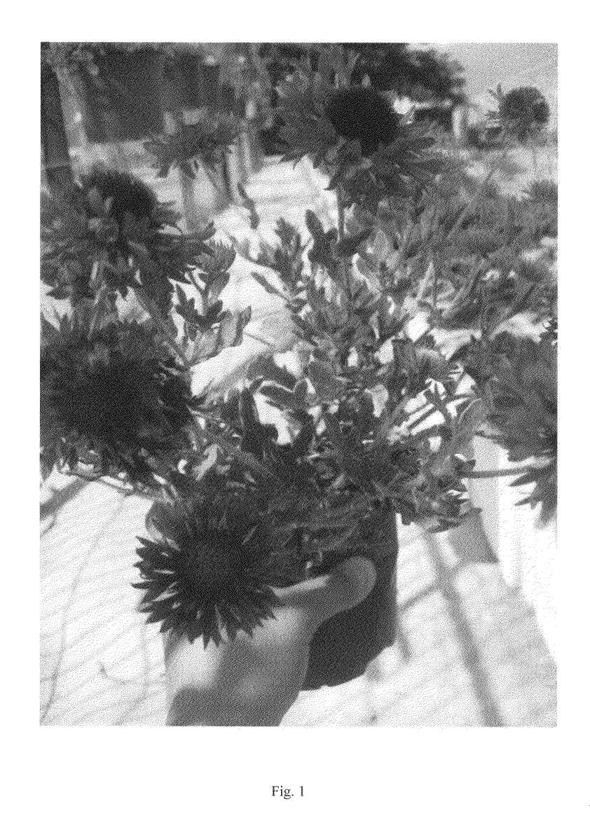

The accompanying photograph in FIG. 1 illustrates in full color a typical plant of ‘DGAL906’ grown in a greenhouse, in a 12 cm pot. Age of the plant photographed is approximately 8 weeks from a rooted cutting. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001 except where general terms of ordinary dictionary significance are used. The following observations and measurements describe ‘DGAL906’ plants grown outdoors during Spring in Moshav Mishmar Hashiva, Israel. The growing temperature ranged from 18° C. to 28° C. during the day and from 13° C. to 18° C. during the night. General light conditions are bright, normal sunlight. Measurements and numerical values represent averages of typical plant types. Plants are approximately 12 weeks old from a rooted cutting.
Botanical classification: Gaillardia pulchella ‘DGAL906’
Propagation:
   Time to initiate roots.—About 4 days at approximately 20° C.
   Root description.—Fleshy, well branched.
Plant:
   Age of plant described.—Approximately 84 days from a rooted cutting.
   Growth habit.—Compact, low growing and slightly spreading.
   Pot size of plant described.—12 cm.
   Height.—Approximately 25 cm.
   Plant spread.—Approximately 45 cm.
   Growth rate.—Moderate.
   Branching characteristics.—Well Branched.
   Length of primary lateral branches.—Approximately 15 cm.
   Quantity of primary lateral branches.—Approximately 20.
   Characteristics of primary lateral branches.—Form: basal Branching Diameter: 0.2 cm. Color: Near RHS yellow green 144A Texture: Densely covered with short bristly hairs Strength: Very strong.
   Internodes length.—Average 1.5-2.0 cm.
Foliage:
   Leaf.—Shape: Pinnatisect Quantity: Approximately 80-100 per branch. Average Length: 5.5 cm. Average Width: 2-2.5 cm. Apex: rounded Base: long attenuate Margin: entire Texture of top surface: very slightly pubescent Pubescence: delicate, not dense Aspect: 45 degrees upwards Color: Young foliage upper side: Near RHS green 137A Young foliage under side: Near RHS green 137B Mature foliage upper side: Near RHS green 137C Mature foliage under side: Near RHS green 138B Venation: Type: Pinnate Venation color upper side: Near RHS green 138B Venation color under side: Near RHS green 138C Petiole: Sessile.
Flower:
   Natural flowering season.—March to August, long day conditions.
   Time to flowering from rooted cutting.—4 to 5 weeks.
   Inflorescence and flower type and habit.—Solitary.
   Rate of flower opening.—3 to 5 days from bud to fully opened flower.
   Flower longevity on plant.—5 to 7 days.
   Persistent or self-cleaning.—Persistent.
   Flower size.—Diameter of entire flower: Approximately 5 cm. Depth of flower: Approximately 1.6 cm. Disc diameter: Approximately 2.4 cm Receptacle shape: Rounded Receptacle height: Average 0.5 cm. Receptacle diameter: Average 1.4 cm. Receptacle color: Near RHS yellow green 144D Quantity of inflorescences per lateral stem: 1-3 Quantity of open inflorescences and buds per plant: 20-30.
   Bud.—Shape: Flattened globular Length: 0.5-0.9 cm Diameter: 1.4-1.8 cm Color: Year RHS Yellow-Green 144A.
   Ray florets.—Quantity: 80 Arrangement: Rotate in a triple whorl Length: 1.8 cm Width: 1.2 cm Shape: Fan-Shaped Apex: Acute Base: fused into tubular shape Margin: Entire Texture, upper and lower surfaces: Smooth, slightly ribbed longwise.
   Color.—When opening, upper surface Red group 53A near center and Red group 46A at the tips When opening, lower surface: Red group 46A near center and Red group 46D RHS at the tips Fully opened, upper surface: Red group 46A near center and Red group 46D at the tips Fully opened, lower surface: Orange-Red group N34A near center and Orange-Red group N34C at the tips.
   Disc florets.—Disc flowers are only rudimentary flowers. Non-ornamental, with reproductive organs present. Quantity: 120 Arrangement: Spirally placed on disc Length: 0.8 cm Width: 0.3 cm Shape: Eliptic Apex: Acute Base: Fused Margin: Entire Texture, upper and lower surfaces: Densely covered with short bristly hairs
   Color.—White group 155A near center and Gray-Purple group 187A at the tips.
   Phyllaries/involucal bracts.—Quantity: Approximately 32 Length: 1.5 cm Width: 0.3 cm Shape (overall): Oblanceolate Apex: Apiculate Base: Truncate Texture, both surfaces: Dull, covered with short hairs Margin: Entire, with short hairs directly on margin Color: Near RHS yellow green 137B.
   Peduncles.—Length: Average 8 -10 cm Diameter: 1.5 cm Angle: About 45° to the lateral branch Strength: very strong Texture: Slightly glossy, somewhat pubescent. Color: Near RHS yellow green 147.
   Fragrance.—Slightly sweet scent.
Reproductive organs:
   Disc florets.—
   Stamens.—Number: 4.
   Anthers.—Shape: Liguate Length: Approximately 0.5mm Color: Greyed-Purple group 187A at the tips Pollen: Present Color: Greyed-yellow group162A Quantity: Moderate.
   Pistil.—Number: One present Length: 10 mm Style: Tubular Length: 4 mm Color: White group 155A near center Stigma: Shape: Bifid Color: Greyed-Purple group 187A at the tips Ovary Color: White group 155A.
   Ray florets.—No reproductive organs present.
Other characteristics:
   Seeds and fruits.—50-70 seeds per flower, Shape — oblong triangle, size — 0.2 cm long×0.15 cm wide, with spiny crown on top 0.3 cm long×0.2 wide.
   Disease/pest resistance.—Neither resistance nor susceptibility to pathogens and pests common to Gaillardia have been observed.
   Temperature tolerance.—The new variety exhibits a good temperature tolerance range for Gaillardia. Known tolerance for high temperatures to at least 35° C. Lower temperature tolerance unknown.

What is claimed is:

1. A new and distinct cultivar of Gaillardia plant named ‘DGAL906’ as herein illustrated and described.
